(19) United States
(12) Patent Application Publication
(10) Pub. No.: US 2025/0194934 A1
(43) Pub. Date: Jun. 19, 2025

(54) METHODS AND SYSTEMS FOR AUTOMATIC ASSESSMENT OF BREAST MORPHOLOGY

(71) Applicant: OpAI Innovations Inc., Montreal (CA)

(72) Inventor: Prabhakara Subramanya JOIS, Montreal (CA)

(73) Assignee: OpAI Innovations Inc., Montreal, QC (CA)

(21) Appl. No.: 18/849,007

(22) PCT Filed: Mar. 20, 2023
(86) PCT No.: PCT/CA2023/050361

§ 371 Date: Sep. 20, 2024

Related U.S. Application Data
(60) Provisional application No. 63/322,366, filed on Mar. 22, 2022.

(51) Int. Cl. A61B 5/00

(52) U.S. Cl. CPC A61B 5/0091; A61B 5/0013; A61B 5/0064; A61B 5/742

(57) ABSTRACT

There are described various methods and systems for automatic assessment of breast morphology using three-dimensional (3D) sensor data of abreast area collected in situ using a time-of-flight (ToF) sensor. The 3D sensor data is processed to determine one or more breast morphology parameters in respect of one or more breasts in the breast area. The methods and systems also provide graphical outputs comprising 3D visualizations of the stacking of the overlayed regions of the breast morphology and “Volumetric and Iso-contour based Morphological Asymmetry” (VIMA) scores for automatically and objectively assessing breast asymmetry.

### CROSS-REFERENCE TO PREVIOUS APPLICATION

This application claims priority from U.S. provisional patent application No. 63/322,366 filed on Mar. 22, 2022, which is incorporated herein by reference in its entirety.

### FIELD

The embodiments described herein generally relate to assessing breast morphology, and in particular, to a system and method for automated assessment of breast morphology, as well as automated assessment of breast asymmetry, and more broadly, automated assessment of the dissimilarity between a desired breast morphology and an existing (or current) breast morphology.

### BACKGROUND

Breast cancer is among the most common forms of cancer, affecting nearly one in every eight women in North America. Treatment for breast cancer is normally dependent on its stage of development, as well as the particular type of affected cancerous tissue. In general, treatments involve one or more of invasive surgery, chemotherapy and/or radiation therapy.

To this end, one of the standard approaches for treating breast cancer is a mastectomy. Mastectomy is a type of invasive surgery involving the complete surgical removal of the affected breast tissue. Although lifesaving, this invasive procedure can have a number of common and significant side-effects. For example, owing to the removed breast tissue, patients may have asymmetric breasts, or otherwise may have an altered breast morphology. This, in turn, may have negative impacts on breast cancer survivors' emotional well-being, body image, and quality of life.

In view of this, present-day treatment for breast cancer encompasses the initial removal of cancerous tissue, subsequently followed by the aesthetic surgical reconstruction and modification of the patient's breast morphology such as to achieve an aesthetically pleasing breast form. Yet, despite the growing demands for aesthetic and reconstructive breast surgeries—until recently, breast morphology and reconstruction outcomes have been primarily evaluated by the surgeon's subjective naked eye assessment, as well as simple anthropometry (i.e., tape measurements). In turn, these surgeries often fall short of effectively restoring the patient's breast aesthetics. Even with the availability of photogrammetry and hand-held 3D depth sensing technology, there is still a dearth of fully automated commercially available perioperative (pre, intra, and postoperative) assessment techniques which objectively and instantaneously (i.e., in real-time or near real-time) assess patients' breast morphology for the purpose of guiding reconstructive and aesthetic breast surgeries.

### SUMMARY OF THE VARIOUS EMBODIMENTS

The following introduction is provided to introduce the reader to the more detailed discussion to follow. The introduction is not intended to limit or define any claimed or as yet unclaimed invention. One or more inventions may reside in any combination or sub-combination of the elements or process steps disclosed in any part of this document including its claims and figures.

In one broad aspect of the present disclosure, there is provided a method for automatic assessment of breast morphology. The method comprises receiving three-dimensional (3D) sensor data of a breast area and processing the 3D sensor data to determine one or more breast morphology parameters in respect of one or more breasts in the breast area. The processing comprises generating a 2D image projection of a curvature response of the 3D sensor data and detecting, in the 2D image, one or more key points. The processing also comprises estimating at least one 2D breast region boundary based on the one or more key points and extrapolating the at least one 2D breast boundary region into 3D space to project the at least one breast boundary region onto a 3D image of the breast area The processing also comprises determining the one or more breast morphology parameters based on the at least one breast region boundary defined in one or more of the 2D and 3D images of the breast area and generating an output comprising the one or more breast morphology parameters.

In some examples, the method initially comprises applying simultaneous localization and mapping (SLAM) to the 3D sensor data to generate point cloud data. The method may also initially comprise processing the point cloud data to generate a 3D mesh image of the breast area, the 3D mesh image corresponding to the 3D image of the breast area and generating the 2D curvature tensor field image based on the 3D mesh image.

In some examples, the key points correspond to a position location of one or more of a right nipple areolar complex (NAC), a left NAC and a sternal notch.

In some examples, detecting the one or more key points in the 2D image further comprises bisecting the 2D image to generate a right sub-image and a left sub-image and, within each sub-image, determining the point of maximum curvature as corresponding to the right NAC and left NAC key points, respectively. Detecting the one or more key points in the 2D image may also further comprise cropping the 2D image to generate a cropped image, wherein the bottom left and right corners of the cropped image are aligned with the right and left NAC key points, respectively. Detecting the one or more key points in the 2D image may also further comprise, within the cropped image, determining the point of maximum curvature as corresponding sternal notch (SN) key point.

In some examples, estimating at least one 2D breast boundary region based on the one or more key points further comprises identifying a respective higher curvature region around each of the right and left NAC key points corresponding to higher curvature response. estimating at least one 2D breast boundary region based on the one or more key points may further comprise fitting an ellipse around the respective NAC key point and the high curvature region, wherein the ellipse fitted around the high curvature region including the right NAC key point defines the right breast region boundary, and the ellipse fitted around the high curvature region including the left NAC key point defines the left breast region boundary.

In some examples, the one or more breast morphology parameters comprise one or more of linear geometric measurements.

In some examples, after determining one or more breast morphology parameters, the method further comprises determining a dissimilarity between a current breast morphology and a desired breast morphology. The determining a dissimilarity between a current breast morphology and a desired breast morphology may comprise generating an iso-contour for at least one breast region and generating N-level curves within the breast region boundary of the at least one breast region. The determining a dissimilarity between a current breast morphology and a desired breast morphology may further comprise for each i-th level curve of the N-level curves, applying a binary mask to generate a foreground region corresponding to the location of the breast region, estimating a bounding box around the foreground region, and a box centroid, to generate a target foreground region; and overlaying a reference foreground region over the target foreground region to generate an overlayed region comprising an overlap area and one or more non-overlap areas, wherein the reference foreground region is associated with the desired breast morphology and the target foregoing region is associated with the current breast morphology.

In some examples, the non-overlap areas comprise one or more of false negative areas and false positive areas, wherein the false negative areas are areas which exist in the reference foreground region and not in the target foreground region, and false positive areas are areas which exist in the target foreground region and not in the reference foreground region.

In some examples, the method further comprises stacking the overlayed regions for each successive i-th level curve to generate a 3D visualization of the overlayed regions.

In some examples, the determining the pseudo-volume of a breast region further comprises adding the areas within the iso-contours from all N-level curves.

In some examples, the method is applied to both the right and left breast regions.

In some examples, the method further comprises determine a degree of asymmetry between the breasts, wherein for each i-th level curve. The method further comprises selecting the foreground region for one of the breast regions as being the target foreground region and reference foreground region and flipping the reference foreground region, across a median line defined with respect to the sternal notch key point, to overlay the target foreground region.

In some examples, the method further comprises determining a “Volumetric and Iso-contour based Morphological Asymmetry” (VIMA) score according to the equation:

$$VIMA = 1 - [ λ * mIoU + ( 1 - λ ) * {V}_{r} ]$$

wherein λ is an empirically determined variable, mIoU is a mean intersection over union (IoU) determined across all N-level curves and Vᵣ is a ratio of breast mound volumes.

In some examples, the 3D sensor data is received from a 3D image sensor.

In some examples, the 3D image sensor is one of a time-of-flight (ToF) and LiDAR sensor.

In some examples, the method initially comprises scanning the breast area of a subject using the 3D image sensor.

In some examples, the 3D image sensor is integrated into a portable user device.

In some examples, the method is performed in real-time or near real-time.

In some examples, the output comprises a graphical output.

In some examples, the graphical output comprises the 3D visualization of the stacking of the overlayed regions for each successive i-th level curve.

In some examples, the 3D visualization comprises, for each overlayed region in each i-th level curve, different visual indicia for the overlap area and the one or more non-overlap areas.

In some examples, the 3D visualization comprises, for each overlayed region in each i-th level curve, different visual indicia for each of the false negative areas and the false positive areas.

In some examples, the different visual indicia correspond to a different color or shading schemes.

In some examples, the output is generated on a display interface associated with a user device or a remote computer terminal.

In some examples, the method further comprises receiving, via an input interface of the user device, a selection of one of the level curves in the 3D visualization and updating, on the display interface, the visualization to show visual indicia for the overlap and non-overlap areas for that level curve.

In another broad aspect of the present disclosure, there is provided a system for automatic assessment of breast morphology. The system comprises at least one 3D image sensor for generating 3D sensor data and at least one processor configured to perform any of the above-described methods.

In yet another broad aspect of the present disclosure, there is provided a non-transitory computer readable medium storing computer executable instructions, which upon execution by at least one processor, cause at least one processor to perform any of the above-described methods.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1 is a simplified block diagram of an example embodiment of a system for automatic assessment of breast morphology;

FIG. 2A is a simplified hardware/software block diagram of an example user device, in accordance with some embodiments;

FIG. 2B is a simplified hardware/software block diagram of an example computing terminal and/or external server, in accordance with some embodiments;

FIG. 3 is a process flow of an example embodiment of a method for automatic assessment of breast morphology;

FIG. 4A is a process flow of an example embodiment of a method for pre-processing three-dimensional (3D) sensor data;

FIG. 4B is a process flow of an example embodiment of a method for automatic determination of various breast morphology parameters;

FIG. 4C is a process flow of an example embodiment of a method for automatic determination of a degree of dissimilarity between target and reference breast iso-contours;

FIG. 5A is an illustration of example point cloud data obtained of a subject's torso using a three-dimensional (3D) sensor;

FIG. 5B is an illustration of an example 3D mesh image generated from the point cloud data of FIG. 5A;

FIG. 5C is an illustration of an example two-dimensional (2D) projection of the curvature response generated from the point cloud data of FIG. 5A;

FIG. 6A are various images illustrating an example process for detecting one or more key points in a two-dimensional (2D) projection of curvature response;

FIG. 6B are various images illustrating an example process for ellipse fitting for defining boundaries for one or more breast regions;

FIG. 7 is an illustration of example linear geometric measurements that may be acquired using detected key points and breast region boundaries;

FIG. 8A is an illustration of iso-contours of breast regions, generated based on point cloud data;

FIG. 8B is an illustration of binary mask generation from the various iso-contours (also referred to herein as level curves) at different iso-values (also referred to herein as levels);

FIG. 8C is an illustration of object detection and estimation of bounding boxes in an example level curve;

FIG. 8D is an illustration of an example process for flipping a reference breast region to overlay a target breast region for an i-th level curve;

FIG. 8E is an illustration of various reference breast regions that are flipped to overlay target breast regions for different i-th level curves;

FIG. 8F is an illustration of stacked level curves which show morphological dissimilarities between overlayed reference and target breast regions; and

FIG. 9 is an illustration of example breast regions re-oriented on to the XY plane.

Further aspects and features of the example embodiments described herein will appear from the following description taken together with the accompanying drawings.

### DESCRIPTION OF VARIOUS EMBODIMENTS

It will be appreciated that numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

The terms “including,” “comprising” and variations thereof mean “including but not limited to,” unless expressly specified otherwise. A listing of items does not imply that any or all of the items are mutually exclusive, unless expressly specified otherwise. The terms “a,” “an” and “the” mean “one or more,” unless expressly specified otherwise.

As used herein and in the claims, two or more elements are said to be “coupled”, “connected”, “attached”, or “fastened” where the parts are joined or operate together either directly or indirectly (i.e., through one or more intermediate parts), so long as a link occurs. As used herein and in the claims, two or more elements are said to be “directly coupled”, “directly connected”, “directly attached”, or “directly fastened” where the element are connected in physical contact with each other. None of the terms “coupled”, “connected”, “attached”, and “fastened” distinguish the manner in which two or more elements are joined together.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example, and without limitation, the programmable computers may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and combination thereof.

Program code may be applied to input data to perform the functions described herein and to generate output information. The output information is applied to one or more output devices, in known fashion.

Each program may be implemented in a high-level procedural or object-oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g., ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

In the description herein, the term “article” is used to refer to an object that is being manufactured, produced, packaged, transported, and/or distributed etc. As used herein, the term “article” may refer to a product and/or a package containing a product. An “article” may refer to a product that is intended to be received/used by a retailer, distributor and/or end-user and/or the entire package that may be received by a retailer, distributor and/or end-user including external packaging and/or containers and the goods/products contained therein.

As discussed in the background, mastectomies (i.e., prophylactic mastectomies) are a common method used for removing existing cancerous tissue in patients with a view to reducing the chance of developing cancer in the future. However, a number of studies have shown compelling evidence of the negative effects of mastectomy procedures on a breast cancer survivor's quality of life, body image and emotional well-being.

To mitigate concerns associated with mastectomy procedures, in recent years, breast reconstructive surgeries have become an important component of the breast cancer treatment process and are used to improve patient self-esteem. Reconstructive surgeries enable molding of the patient's breasts to provide better breast symmetry, as well as more generally, to improve the patient's breast morphology. Any dissatisfaction with the surgical outcome of the reconstructive surgery is often addressed with follow-up revisional surgeries where the surgeon will attempt to modify the post-reconstructive breast morphology until the patient is satisfied with the outcome.

In conducting reconstructive or revision-based surgeries, surgeons will often adjust the patient's breasts based only on a naked eye assessment and a qualitative evaluation of the patient's existing breast morphology. The surgical outcome is therefore heavily dependent on the surgeon's subjective experience, as well as their innate ability in adjusting the breast to the desired outcome. Less skilled surgeons may, therefore, generate poorer reconstructive or revised results for patients.

In view of this, there has been a greater demand to replace subjective standards with more objective and quantitative standards for assessing breast morphology. To this end, examples of existing “objective” standards for assessing breast morphology include various manual techniques. These techniques include, for example, the use of direct anthropometry, or simple tape-based measurements of the subject's breasts to determine and adjust morphology parameters. These methods, however, are often inaccurate.

Other example manual techniques include the use of the water test. In a water test, the subject's breast mounds are individually inserted into water containers. The approximate volume of the breast is determined based on the amount of water displaced from the water container. The water test, nevertheless, is both impractical in a real life setting during and after surgery, and can only provide volumetric measurements, i.e., as opposed to shape and position measurements.

Still another common manual technique is the use of mesh/plaster cast. This involves physically pressing a cast against the subject's torso area in a careful manner to procure an inverted mold of the torso. The inverted mold is used to quantify the subject's breast morphology. However, the use of mesh/plaster case is both time consuming and non-feasible in an intra-operative setting, and during surgery.

More generally, where the analysis is performed manually, the analysis is often laborious, time consuming, subjective, and may consume numerous clinical hours. Further, clinically significant variations may exist in the manual assessment because of the differences in how individual surgeons perform the assessment.

With the advancements in medical image processing and computer vision, there has now been a growing interest in replacing manual techniques with software-based imaging techniques. These software-based imaging techniques can be used for morphological assessment of breasts during surgical planning. The imaging techniques include, for example, three-dimensional (3D) scanning through image-photogrammetry. In particular, the non-invasive nature of medical imaging techniques makes data acquisition and processing relatively easy and makes it possible to compute approximate values for breast measurements, size and volume more accurately.

Still, current imaging techniques suffer from important drawbacks. For example, the use of photogrammetry is known to consume a lot of time before producing 3D data of the patient's breasts. Further, existing imaging techniques lack full automation. For instance, current imaging techniques require identification of landmark points in the imaged breasts in order to generate linear measurement to assess breast morphology. The selection of the landmark points is often not automated and must be manually selected by trained personnel before performing any assessment. Many imaging methodologies, therefore, still require manual intervention, prior to producing linear measurements. In turn, these assessments are unable to provide automated, and instantaneous assessments of breast morphology.

Many existing imaging techniques also suffer from the inability to capture intraoperative data needed for assessment, while the patient is in the supine position. Rather, in many imaging methods, the breast profiles are initially acquired while the patient is in the standing position and using non-portable 3D scanners. In turn, these methods are not viable in intraoperative settings when the patient is likely in the supine, rather than standing position. The acquired data is then typically fed into software package(s) on dedicated desktop platforms that are often constrained by driver and connectivity issues. In many cases, this introduces considerable overhead during data transmission and assessment, causing logistical and technical issues in clinical settings.

Still further, existing imaging techniques also fail to provide real-time instantaneous solutions for breast asymmetry assessment, and are only used for semi-automated surgical planning, visualization and 3D measurement. Hence, these techniques are not viable in a perioperative (mainly intraoperative) settings during surgery. In turn, these encourage—rather than dissuade—surgeons to only rely primarily on their experience and naked eye assessment.

In view of the foregoing, and to at least partially mitigate challenges associated with existing systems, embodiments herein provide for a method and system for automatic and quantitative assessment of breast morphology.

As provided in greater detail herein, the disclosed embodiments enable assessment of breast morphology using imaging techniques. In at least one embodiment, a 3D sensor (i.e., a time-of-flight sensor and/or LiDAR) is used to screen a subject's torso area, i.e., including the breast area. The 3D sensor can be a stand-alone device or may be incorporated as part of a portable user device. Acquired 3D sensor data is then automatically processed to determine the subject's breast profile and/or morphology parameters, for one or both of the subject's breasts.

In at least some embodiments, the 3D sensor data can also be processed to determine a degree of dissimilarity between the subject's current 3D breast profile (or morphology) and a desired breast profile. For example, the 3D sensor data can be analyzed to determine the degree of dissimilarity between the subject's current breast profile, and a desired extraneous reference breast profile. In other cases, the 3D sensor data can be analyzed to determine the degree of dissimilarity between the subject's left and right breast profiles, i.e., the degree of asymmetry.

In accordance with some teachings herein, once the 3D sensor data is processed, a graphical output may be generated. The graphical output (i.e., image and/or text) may summarize and visualize the various determined breast morphology parameters. The graphical output may also comprise a visualization of the discrepancy between the desired and target breast profiles. To this end, the graphical output may act as an assistive and informative guide tool aid for medical practitioners. For example, the graphical output can act as an assistive tool in pre-operative settings, and during surgical planning, to better understand the subject's breast morphology vis-à-vis a desired morphology. The graphical output can also act as an assistive tool in post-operative settings, to quantitatively assess surgical outcomes.

In at least some cases, 3D sensor data may be acquired and processed in real-time or near real-time. In turn, the graphical output may also be updated in real-time or near real-time, as the subject's breast profile is being modified and updated during surgery. Accordingly, in intra-operative settings, the graphical output can also act as a real-time or near real-time guide for medical practitioners performing surgery.

Accordingly, in contrast to existing systems for assessing breast morphology, the disclosed embodiments provide for a fully automated objective assessment of breast morphology, which may be performed in real-time or near real-time. As the subject's torso area can be scanned using a portable user device, the disclosed methods and systems can be applied irrespective of whether the subject is in the supine or standing position, and therefore can be used in peri-operative settings (i.e., pre-, intra- and post-operative settings).

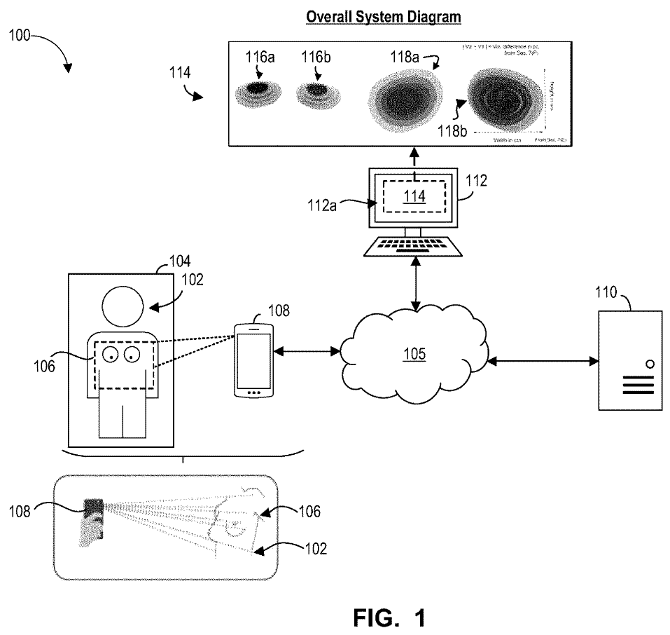

Reference is now made to FIG. 1, which shows an example embodiment of a system 100 for automatic assessment of breast morphology, or breast profile, in accordance with at least some embodiments.

As shown, system 100 generally includes a user device 108 coupled, via network 105, to one or more of an external server 110 and/or an external computing terminal 112.

User device 108 may be a hand-held portable device, which may be used for scanning a patient's 102 torso area 106, as explained in greater detail herein. User device 108 may comprise, for example, a cell phone with integrated or connected scanning functionality, or otherwise any other specialized or non-specialized scanning device. User device 108 may also comprise any other non-portable device, i.e., a standard desktop computer with integrated or connected scanning functionality.

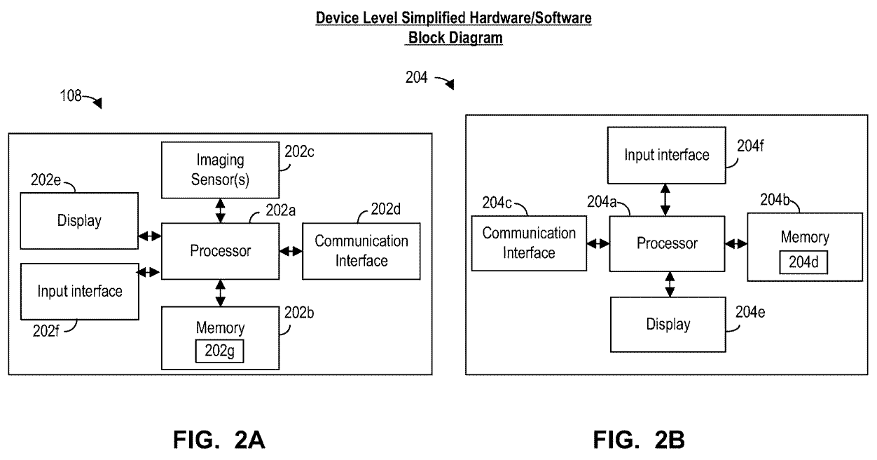

As explained in greater detail herein with reference to FIG. 2A, the user device 108 may comprise a processor 202a in communication with one or more of a memory 202b, one or more three-dimensional (3D) imaging sensors 202c, a communication interface 202d, a display 202e and an input interface 202f. The 3D imaging sensor 202c may comprise, for example, a time-of-flight (ToF) camera or other LiDAR based sensor technology.

As shown, the portable user device 108 may be positioned such that the subject's front torso area is within the frame of view of the sensor 202c. The sensor 202c is then operated to capture 3D sensor data (i.e., point cloud data) in respect of the subject's torso area. As stated previously, an appreciated advantage of using the portable user device is that the patient's torso area may be scanned while the patient is in the supine position. That is, in contrast to existing solutions, the disclosed systems and methods are not limited to acquiring 3D data while the patient is in a standing position. Accordingly, scanning may be performed in intra-operative settings, while the patient is lying on a surgical table.

As shown in FIG. 2A, the user device memory 202b may store a scanning software application 202g. Scanning application 202g can be operated by a device user to cause the imaging sensor(s) 202c to acquire 3D sensor data. For example, the scanning software 202g may generate a graphical user interface (GUI), i.e., on the display 202e, to enable user control via input interface 202f.

As provided herein, the 3D data generated by the 3D imaging sensor 202c may be processed to determine one or more breast morphology parameters. As used herein, breast morphology (or breast profile) parameters may refer to the shape, size, position, asymmetry and appearance—or generally any other geometric feature—of one or more of the subject's breasts. Breast morphology parameters can also refer to various geometric features of an artificial breast mound, i.e., which is scanned by the user device 108.

Three-dimensional (3D) data, acquired by the imaging sensor 202c, can also be processed to determine a level of dissimilarity between a current breast profile and a desired breast profile. For example, the subject's breast profiles can be compared to an extraneous reference (or desired) breast profile to determine a level of dissimilarity. In other cases, the acquired 3D data can also be analyzed to determine the degree of dissimilarity between the profile or morphology of each of the subject's breasts. This, in turn, can assist in determining the degree of asymmetry between the subject's breasts.

In some embodiments, the processing of the 3D sensor data may occur on one or more of the external server 110 (i.e., a cloud server), or otherwise, the external computing terminal 112 (i.e., a desktop computer). For example, upon acquisition of the 3D sensor data, the 3D sensor data may be transmitted in raw form, or at least partially processed form, to one or more of the server 110 and/or computer terminal 112, via network 105, for processing.

To this end, network 105 may be connected to the internet. Typically, the connection between network 105 and the Internet may be made via a firewall server (not shown). In some cases, there may be multiple links or firewalls, or both, between network 105 and the Internet. Some organizations may operate multiple networks 105 or virtual networks 105, which can be internetworked or isolated. These have been omitted for ease of illustration; however, it will be understood that the teachings herein can be applied to such systems. Network 105 may be constructed from one or more computer network technologies, such as IEEE 802.3 (Ethernet), IEEE 802.11 and similar technologies.

With reference to FIG. 2B, each of the server 110 and/or computer terminal 112 may include a respective processor 204a in communication with one or more of a memory 204b and a communication interface 204c. Memory 204b can store, for example, data processing software 204d (i.e., a breast morphology assessment software 204d), which can process 3D sensor data to determine breast morphology parameters. In some cases, the processor 204a may also couple to a display device 204e.

In other cases, the data processing software 204d may be partially or entirely hosted directly on a memory 202b of user device 108. In these case, processing of the 3D sensor data may be partially or completely performed directly on the user device 108.

In at least one embodiment, system 100 may generate various graphical visualization outputs 114 (i.e., images and/or text). Graphical outputs 114 may be displayed, for example, on a display screen 112a associated with the external computer 112 (i.e., display 204e in FIG. 2B) and/or user device 108 (i.e., display 202e in FIG. 2A). In some cases, graphical output 114 may be generated by the breast morphology assessment software 204d.

In more detail, graphical outputs 114 can include visualizations of various breast profile parameters, as determined by analyzing the acquired 3D sensor data. As provided herein, the graphical output 114 can act as an assistive tool guide for users (i.e., medical practitioners) to understanding the geometric features of the subject's breasts.

Graphical output 114 can also include various images, which visually illustrate the morphology or profile of the patient's breasts. For example, images 116a, 116b and images 118a, 118a can correspond to 3D and XY plane projection views of the subject's right and left breasts. As shown, at least one of the displayed images 116, 118—i.e., in graphical output 114—can display the dissimilarity between the subject's current breast profile and a desired or reference breast profile. For instance, images 116b, 118b provide a color or shaded coded overlay between the current breast profile and a reference breast profile and localizing the areas of overlap and non-overlap (i.e., false-positives and false-negatives) between the two breast profiles. In the exemplified embodiment, the dissimilarity is illustrated between the subject's left and right breast profiles, such as to visualize the degree of asymmetry between the breasts. In particular, the right breast profile is selected as the reference profile with a view to illustrating the degree of dissimilarity with the left breast profile. In this example, a surgeon may desire to mold the left breast profile to better resemble the right breast profile (i.e., the reference profile), so as to produce greater symmetry between the two breasts.

In at least one embodiment, the graphical output 114 can be updated in real-time, or near real-time. For example, the user device 108 (or otherwise any 3D imaging sensor), may be positioned to capture 3D sensor data in real-time or near real-time. For example, as a surgeon is performing a breast reconstructive surgery and/or readjusting the breast profile, the user device 108 may perform continuous background scanning of the subject's torso area 106. The generated 3D sensor data may then be processed, in real-time or near real-time, to update the graphical output 114. In this manner, the updated graphical output 114 can act as a real-time, or near real-time, guide for the progress of the surgery. For example, a surgeon can track the changes to the breast profile in real-time or near real-time, via graphical output 114. In other cases, the surgeon can refer to the graphical output 114 to track the degree of dissimilarity between the target and current breast profiles, also in real-time or near real-time.

In some cases, in addition to or in the alternative to providing a graphical output 114, any other suitable output can be generated (i.e., an audio output).

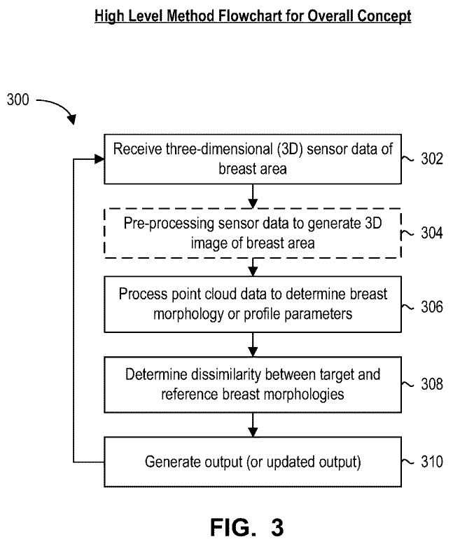

Reference is now made to FIG. 3, which shows a process flow for an example embodiment of a method 300 for automatic assessment of breast morphology. Method 300 can be performed by one or more of the user device processor 202a and/or the processor 204a of the computing device 112 and/or external server 110.

As shown, at 302, three-dimensional (3D) sensor data of the subject's torso area, which includes the breast area (i.e., one or both of the subject's breasts), is acquired. For example, the 3D sensor data may be acquired using the 3D imaging sensor 202c, of user device 108. As discussed previously, the imaging sensor 202c may comprise a range imaging camera, such as a time-of-flight (ToF) camera.

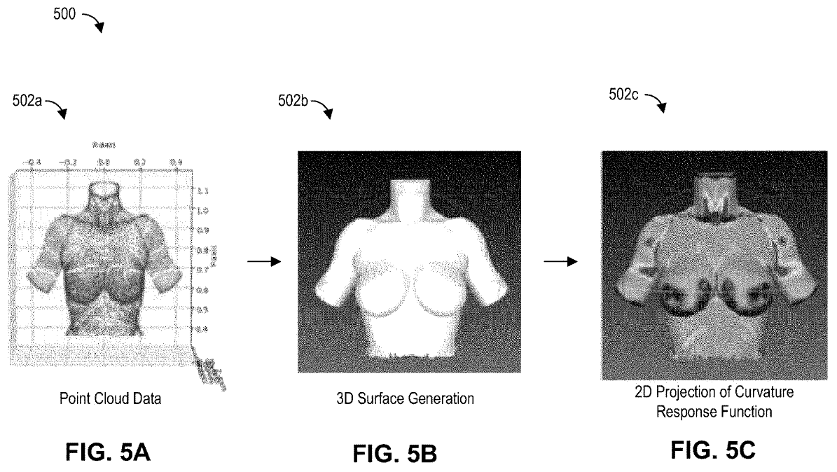

At 304, the 3D sensor data may be initially pre-processed to generate point cloud data (FIG. 5A). Point cloud data is a spatial representation of collected data points and may be used for representing the 3D morphology of the subject's breasts. In at least one embodiment, the point cloud data is generated, and updated (i.e., in real-time or near real-time), by solving a simultaneous localization and mapping (SLAM) problem, which correlates acquired 3D sensor data points to XYZ spatial positions.

At 306, the point cloud data is further processed to determine various breast morphology or breast profile parameters. As indicated previously, the breast morphology (or breast profile) parameters may refer to the shape, size, position, asymmetry and appearance—or generally any other geometric feature—of one or more of the subject's breasts.

At 308, in some embodiments, the breast morphology parameters can also be used to determine a degree of dissimilarity between a reference (or desired) breast profile and a current breast profile, as explained in greater detail herein. In some cases, a specialized score can also be determined, which can indicate the level of asymmetry between the subject's breast morphologies based on the dissimilarity between the two breast profiles.

A 310, an output may be generated based on the data processing at acts 306 and/or 308. For example, as stated previously, a graphical output (i.e., output 114) can be generated on a display of the user device 108 and/or a display of the external computer 112. In some embodiments, the graphical output can display various determined breast morphology parameters, a visual representation of one or more of the subject's breast profiles, and/or a visualization of the dissimilarities between the subject's current breast profile and a reference breast profile.

As also stated previously, in at least one embodiment, the system 100 can be used to monitor, in real-time or near real-time, changes in breast profile or morphology. For instance, as a surgeon is performing a breast reconstructive or revision procedure, the graphical output may be also updated, in real-time or near real-time, to reflect real-time or near real-time changes to the patient's breast morphology. Accordingly, in at least some embodiments, method 300 may iterate such that the output, at act 310, is updated based on newly received 3D sensor data. For example, as the user device 108 is positioned in the background to receive 3D sensor data, in real-time or near real-time—acts 302 to 308 may iterate continuously, in real-time or near real-time, to generate updated outputs at 310.

In other embodiments, method 300 may not necessarily be performed in real-time or near real-time but may only iterate in response to a trigger event. For example, the trigger event may involve detecting a change in the received 3D sensor data. For instance, the system can compare point cloud data received in a previous iteration of method 300, with newly received point cloud. If a change is detected, this can indicate a change in the patient's breast morphology. Accordingly, method 300 can be triggered to re-iterate to generate an updated output 310. In other cases, the trigger event may be a temporal event. For example, method 300 can only iterate after determining that a pre-defined period of time has lapsed from the prior iteration. That is, the method may only iterate at desired pre-defined time or frequency intervals. In still other embodiments, method 300 can be performed offline, based on previously collected 3D sensor data.

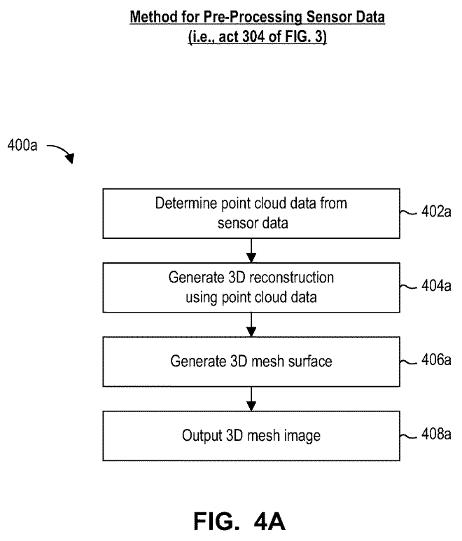

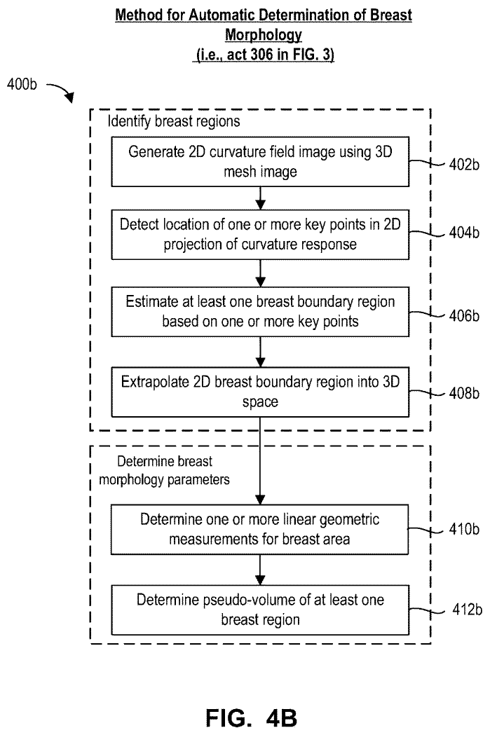

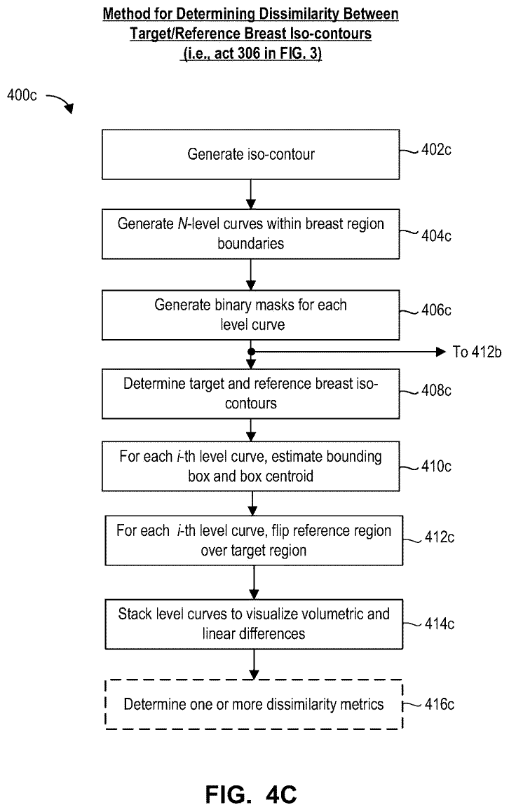

Reference is now made to FIG. 4A, which shows a process flow for an example method 400a for pre-processing 3D sensor data to generate a 3D mesh image of a subject's breast area. Method 400a expands on act 304 in method 300 of FIG. 3.

As shown, at 402a, point cloud data is generated from the sensor data received from imaging sensors 202c, of user device 108. The point cloud data can be used to represent the X, Y, and Z geometric coordinates of each collected sensor data point (see e.g., 502a in FIG. 5A).

At 404a, a 3D reconstruction of the subject's breasts is generated, based on the point cloud data. In at least one embodiment, the 3D reconstruction is generated by solving a simultaneous localization and mapping (SLAM) problem. In various cases, SLAM is applied during data acquisition as the sensor data is being received by the system processor.

At 406a, a 3D mesh surface is generated based on the generated 3D reconstruction (see e.g., image 502b in FIG. 5B). In some embodiments, the 3D mesh surface is generated by applying a marching cubes algorithm to the reconstructed point cloud data. The marching cubes algorithm extracts a polygonal mesh surface from the discrete 3D points.

At 408a, a 3D mesh surface image is output, which can be used to assess various breast morphology and/or asymmetry parameters. In some embodiments, the 3D mesh image is further cropped, and center aligned, to focus on the subject's breast area. This process can be facilitated by initially constraining the field of view of the scannable area to focus primarily on the subject's breasts at act 302 of method 300.

Reference is now made to FIG. 4B, which shows a process flow for an example method 400b for processing 3D sensor data to determine various breast morphology parameters. Method 400b expands on act 306 in method 300 of FIG. 3.

As shown, the process flow in method 400b may be generally segmented into two sub-process flows, namely: (i) initially, analyzing the 3D mesh image (e.g., image 502b in FIG. 5B) to identify each of the subject's breast regions (acts 402b-408b); and (ii) subsequently, determining the breast morphology parameters by analyzing the identified breast regions (acts 410b-412b). A curvature response of the 3D mesh image (i.e., 3D sensor data) can then be estimated using known mean and gaussian curvature filters.

In more detail, at 402b, the 3D mesh image—generated at act 408a of method 400a—is processed to generate a two-dimensional (2D) projection of the 3D mesh image's curvature response from the subject's breast area, whereby the depth and curvature (i.e., mean and gaussian curvature) are represented by different indicia, i.e., different pixel color values of the 2D image (see e.g., image 502c in FIG. 5C). In this manner, the 3D image is reduced to a 2D image, all the while preserving the 3D features via the curvature response data. This, in turn, may simplify processing and analysis of the subject's breast area. In at least one embodiment, act 402b may be performed using Python® or Meshlab®, and in accordance with the techniques disclosed in Goldgof D B, Huang T S, Lee H. “A Curvature-Based Approach to Terrain Recognition”, IEEE Transactions on Pattern Analysis and Machine Intelligence, 1989 November; 11(11):1213-7.

At 404b, one or more key points are detected from the 2D projection of the curvature response image. The key points correspond to specific areas (or locations) in the subject's breast area. As explained herein, key points are used for localizing the area corresponding to the subject's left and right breast regions within the 2D image, such as to enable determining breast-specific morphology. In at least one embodiment, the identified key points correspond to the subject's sternal notch, as well as one or more of their nipple areolar complexes (NACs).

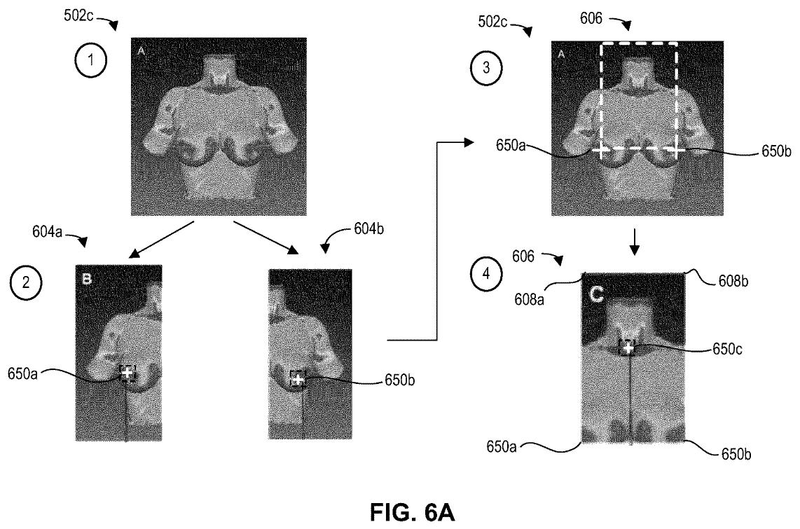

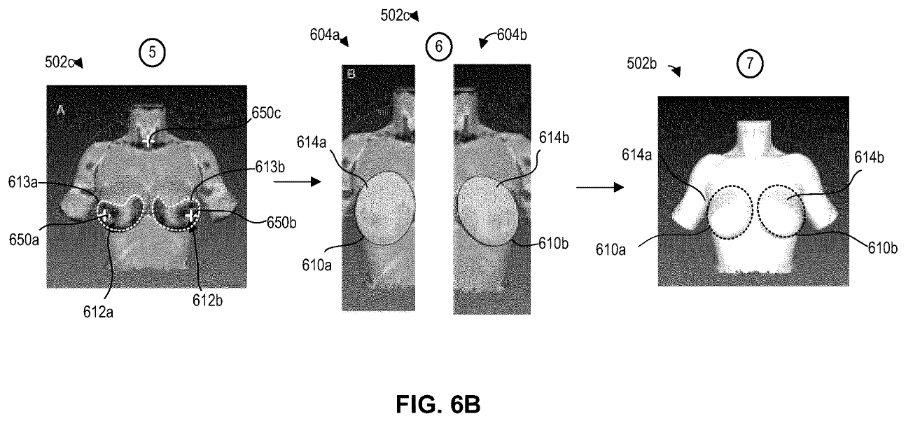

Reference is now briefly made to FIGS. 6A-6B, which visualize an example process for identifying key points at act 404b. As shown, in at least one embodiment, the key points are identified by initially bisecting the 2D curvature image 502c to generate corresponding sub-images 604a, 604b. Sub-images 604a, 604b generally correspond to the area around the right and left breast regions, respectively.

Within each sub-image 604a, 604b, key points—corresponding to the nipple areolar complex (NAC)—are identified. These key points are identified by determining the points with the highest curvature response, within each sub-image 604a, 604b. In the illustrated embodiment, this corresponds to key point 650a for the right breast NAC, and key point 650b corresponding to the left breast NAC. In some embodiments, the point of maximum change of curvature is determined via thresholding the curvature field in each sub-image. For example, this may be performed using an Otsu's multi-thresholding technique having a hyper parameter setting to select the number of threshold levels (i.e., two threshold levels).

Once the NAC key points 650a, 650b are identified, these key points are then used to identify the final key point, corresponding to the subject's sternal notch (SN). The SN key point 650c is identified by cropping the 2D curvature image 502c to generate a cropped image 606. The boundaries of the cropped image 606 are defined by the NAC key points 650a, 650b. For example, as illustrated, the bottom left corner of the cropped 2D projection image 606 can correspond to the right NAC key point 650a, while the bottom right image corner can correspond to the left NAC key point 650b. The top right and left image corners 608b, 608a may align with the boundaries of the original 2D image 502c. In at least one example, the cropped image 606 may have a rectangular shape.

The third key point 650c, corresponding to the sternal notch, is then detected, within this cropped image 606. The sternal notch key point 650c may be detected by again identifying the point of maximum curvature (i.e., highest curvature) within the cropped image field 606. For example, this may also be performed by thresholding the curvature field within the area of the cropped image 606 (i.e., using an Otsu's multi-threshold algorithm).

Referring back to FIG. 4B, at 406b, once the key points 650a-650c are detected, the key points are then used for localizing the subject's left and right breast regions, as well as estimating the boundary region for one or more of the subject's breasts.

To further clarify act 406b, reference is briefly made to FIG. 6B, which illustrates an example process for localizing the right and left breast region boundaries 610a, 610b within the 2D curvature image, based on detected key points 650a-650c.

As shown, in at least one example embodiment, the breast boundary regions 610a, 610b may be determined using an ellipse fitting method (e.g., analogous to that disclosed in H. Sridhar, J. R. Harish Kumar, S. Jois and C. S. Seelamantula, “An Unconstrained Ellipse Fitting Technique And Application To Optic Cup Segmentation,” 2018 IEEE Global Conference on Signal and Information Processing (GlobalSIP), 2018, pp. 514-518, doi: 10.1109/GlobalSIP.2018.8646446). In particular, using the bisected images, 604a, 604b—an ellipse may be drawn around each of the NAC key points 650a, 650b, to define the respective right and left breast regions.

The ellipse fitting method is applied to the regions 612a, 612b from the output of a threshold operation (i.e., Otsu's multi-threshold operation) applied on the 2D curvature projection, which identifies regions 612a, 612b as being of highest curvature around each NAC key point 650a, 650b corresponding to each breast (see e.g., regions 612a, 612b).

An ellipse may then be drawn around each NAC key points 650a, 650b, and using the boundary lines 613a, 613b, delineating regions 612a, 612b, as the edge of the ellipse. The fitted ellipses may accordingly define a right breast boundary edge 610a, defining the right breast region 614a, as well as a left breast boundary edge 610b, defining a left breast region 614b.

Continuing reference with FIG. 4B, at 408b, the 2D breast boundary region—generated at act 406b—is extrapolated back into 3D space. This is shown, for example, in FIG. 6B, whereby the right and left breast region boundaries 610a, 610b are projected back onto the 3D mesh image 502b. This is performed by taking the elliptical boundary defined, on the thresholded curvature response image (i.e., in 2D Euclidean space or XY coordinates), and extrapolating this to 3D space by finding the Z-axis values in the point cloud data for the corresponding XY coordinates along the elliptical boundary.

Once act 406b is completed, the breast boundary regions are defined in both 2D and 3D space, and the remaining portion of method 400b involves determining one or more breast morphology parameters, for one or both of the localized breast regions.

In more detail, at 410b, one or more linear geometric measurements (i.e., standard anthropometry measurements) are determined for one or more of subject's breast regions. In at least one embodiment, these linear geometric measurements are determined using the 2D image projection and based on a combination of: (i) the detected key points 650a-650c, (ii) as well as the elliptical breast boundaries 610a, 610b. To this end, as act 410b relies only on the 2D image projection (rather than the 3D image), act 410b may also be performed prior to, or at least partially concurrently with, act 408b.

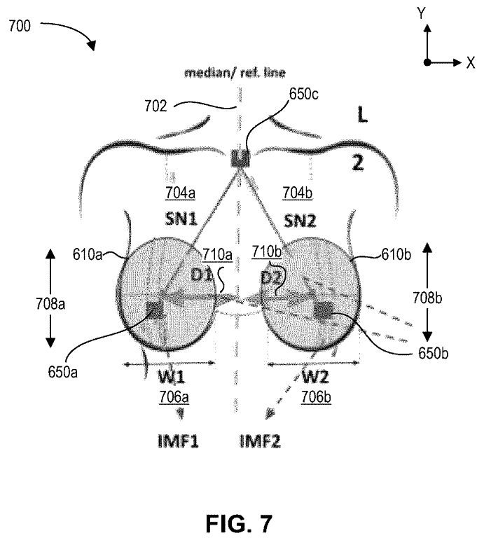

FIG. 7 shows example linear geometric measurements which can be determined at act 410b. As shown, a longitudinal axis 702 may be defined as intersecting the sternal notch key point 650c, and may define the median or reference line, separating the breast region boundaries 610a, 610b. The determined linear measurements can include, by way of non-limiting examples: (i) sternal notch to nipple distances 704a, 704b, which are calculated based on the distance between the sternal notch key point 650c, and each of the NAC key points 650a, 650b, respectively; (ii) breast widths 706a, 706b, which can be calculated by measuring a lateral distance—along an axis orthogonal the longitudinal axis 702, and intersecting the respective NAC key point 650a, 650b—from one end of the respective breast boundary 610a, 610b to the axially opposite end; (iii) breast heights 708a, 708b, which can be calculated by measuring a longitudinal distance—along an axis parallel to axis 702, and intersecting the respective NAC key point 650a, 650b—from one end of the respective breast boundary 610a, 610b to the axially opposite end; and (iv) distances 710a, 710b between each nipple-areola complex (NAC) key point 650a, 650b and median line 702, measured along an axis orthogonal the longitudinal axis 702 and intersecting the respective NAC key point 650a, 650b.

With continued reference to FIG. 4B, at 412b, a pseudo-volume of each, or at least one breast region, is determined. In various cases, this act is performed after act 406c in method 400c.

Reference is now made to FIG. 4C, which illustrates a process flow for an example method 400c for determining the dissimilarity between a current breast morphology and a reference (or desired) breast morphology. Method 400c may also be performed in the course of act 308 in method 300 of FIG. 3.

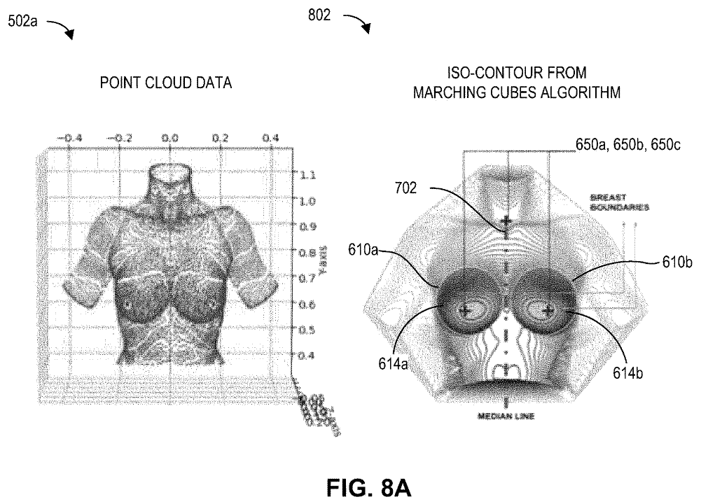

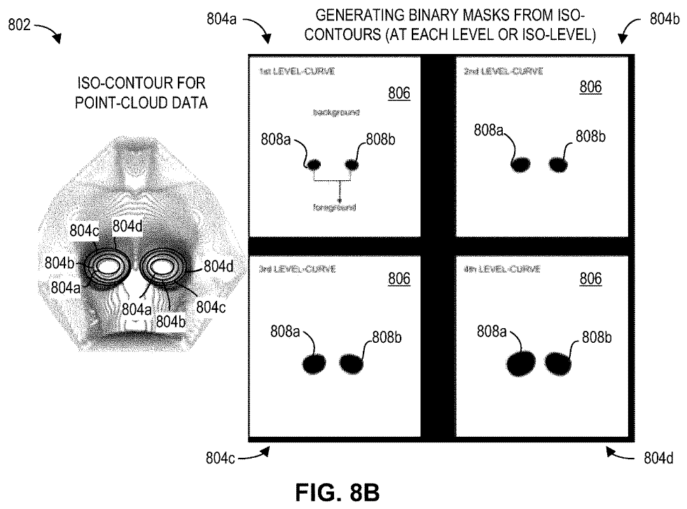

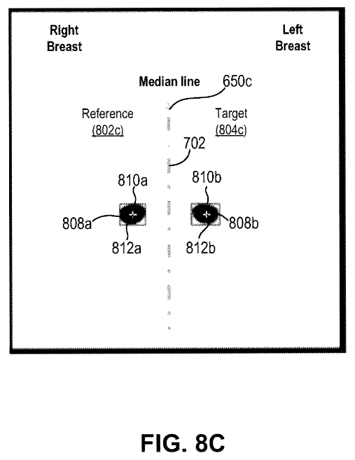

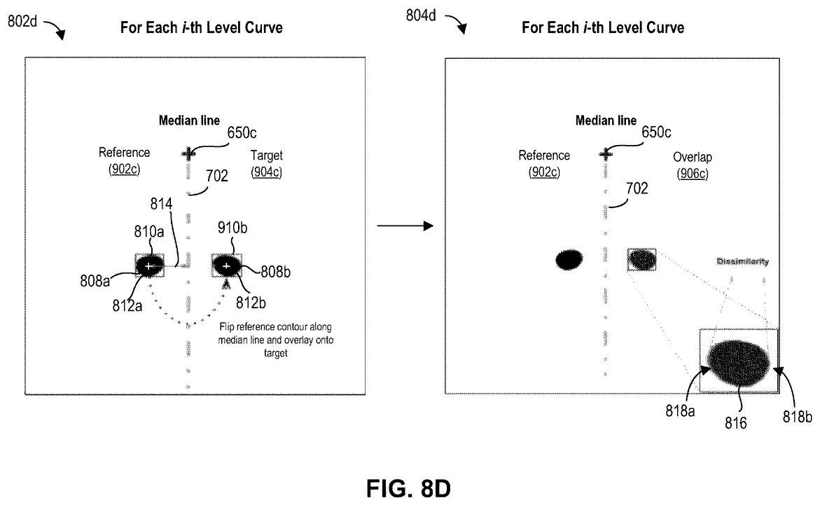

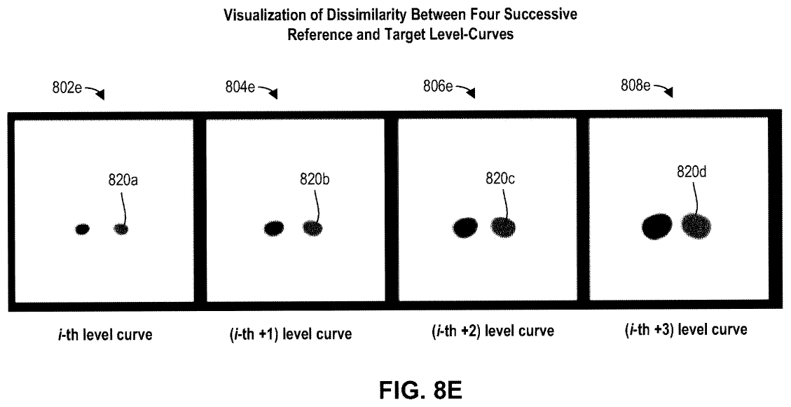

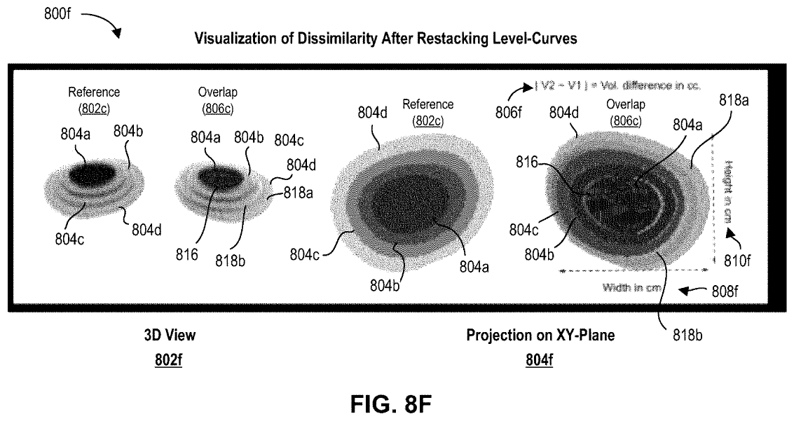

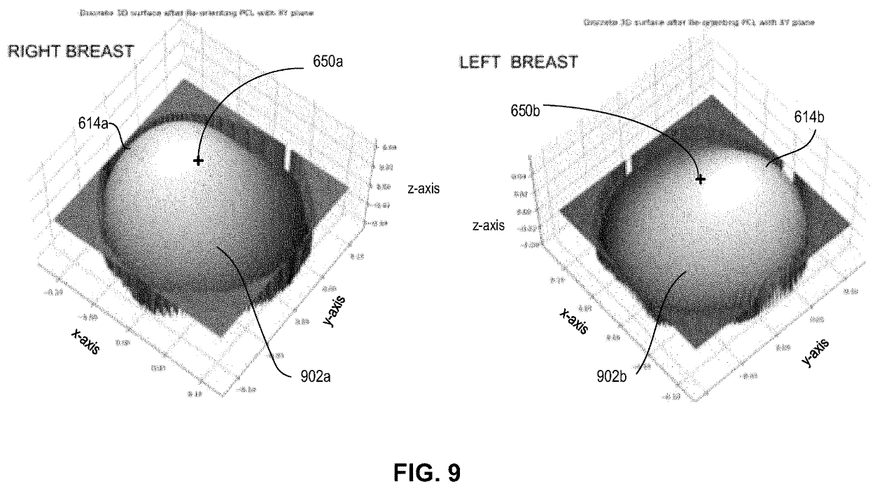

At 402c, an iso-contour (also referred to herein as a level-curve) may be generated for one or more of the right and left breast regions 614a, 614b (e.g., 802 in FIG. 8A). The iso-contour may be determined using the underlying point cloud data 502a and is generated at each iso-value or height defined along the Z-axis (as seen in FIG. 8A), using the marching squares algorithm (i.e., a 2D variant of the marching cubes algorithm). With the orientation of the breast portions 902a, 902b in the XY plane (as seen in FIG. 9).

At 404c, the iso-contours is used to generate any desired number of level curves (i.e., N-level curves), whereby each level curve cuts the re-oriented 3D breast surface (i.e., which is a continuous function), at intervals along the Z-axis (see e.g., level curves 804 in FIG. 8B). The N-level curves are determined along the Z-axis up to the respective key point 650a, 650b (FIG. 8A), and within the respective defined breast boundary regions 610a, 610b. Each level curve corresponds to a cross-section of the 3D surface at the i-th level (iso-value) and corresponds to a 2D cross-sectional image along the XY plane. To this end, the 0^{th} level curve overlaps with the breast boundary and defines the lowermost part of the breast mound.

At 406c, for each level curve, a binary mask is generated. For example, as shown in FIG. 8B, for each example level curve 804a-804b, a binary mask is generated such that the background 806 is discarded, while only the contents within the curve (i.e., within the respective breast boundary 610a, 610b) is chosen as foreground 808a, 808b. Accordingly, for each i-th level curve, one or more foreground regions 808a, 808b are defined, corresponding to the location of the breast region at that i-th level curve.

In some cases, once act 406c is completed, act 412b of the method 400b may be performed. The pseudo-volume of each breast region may be determined by summation of the of the areas of the respective 2D foregrounds (808a or 808b) from all N-levels. That is, at 412b-—for each level curve, and for each breast mound—an area is determined for the respective foreground region. Subsequently, the areas for all foreground regions in all level curves, for a given breast mound, are summed to determine the pseudo-volume for that breast mound.

At 408c, “target” and “reference” breast iso-contours are defined. The “reference” breast iso-contour may correspond to the ideal, or desired, iso-contour for a particular breast. In contrast, the “target” breast iso-contour may be the breast iso-contour which is desired to resemble the reference iso-contour.

For example, in a simplified example, the objective may be to achieve symmetry between the subject's left and right breasts. Accordingly, the “reference” breast iso-contour may be selected as one the left or right breast iso-contours, while the “target” iso-contour may be selected as the opposite breast iso-contour. For example, the “reference” breast iso-contour may be selected as the right breast, while the “target” breast iso-contour may be selected as the left breast. As explained herein, based on the selected reference and target, the system may determine the dissimilarity between the “reference” and the “target” iso-contour. This, in turn, may assist a user (i.e., medical practitioner) to determine how to better operate on the “target” breast (i.e., the left breast) to achieve better symmetry with the “reference” breast (i.e., the right breast).

In another example, the “reference” breast iso-contour may not be one of the subject's breasts but may be an external reference iso-contour. For example, rather than achieving symmetry between the breasts—the objective may be to reshape one or both of the subject's breasts to resemble some idealized breast morphology. The external reference iso-contour may be generated, for example, using a modelling software. Alternatively, the user may also have access to a physical breast mound, representing the reference morphology. The breast mound may be scanned using the user device 108, and an iso-contour may be generated in accordance with methods 400a, 400b as well as act 402c of method 400c. That is, rather than applying the previously described methods to the subject's actual breast to generate an iso-contour, the same methods and principles can be applied to an extraneous breast mound to generate a reference iso-contour. In this example, the target breast iso-contour may comprise one or both of the subject's breasts.

In at least some cases, the reference and target iso-contours may be selected beforehand by a user of the user device 108 and/or computing terminal 112, i.e., using an input interface of these devices. For example, prior to completing the surgery—or otherwise after acquiring the 3D sensor data using the user device 108—a medical practitioner may be presented with a graphical user interface (GUI) through which they may select the target and reference breasts. In other cases, the target and reference iso-contours may be automatically selected by the software program. For example, the software may be pre-configured to select a particular breast iso-contour as the reference and/or target breasts.

In the remaining discussion provided herein, and only for ease of explanation, the left breast iso-contour is selected as the “target” iso-contour, while the right breast iso-contour is selected as the “reference” iso-contour (i.e., 802c and 804c in FIG. 8C). Accordingly, the remaining discussion focuses on an example application where it is desired to determine the degree of asymmetry between the subject's breasts.

At 410c, and as best shown in FIG. 8C, for each i-th level curve, an object detection algorithm is executed to: (i) detect the foreground regions 808a, 808b, corresponding to the breast locations; (ii) estimate a bounding box 810a, 810b around each respective foreground region 808a, 808b; and (iii) determine the location of the centroid for each of bounding box 810a, 810b (i.e., the centroid (Cᵣ) 812a for the reference breast region 808a, and the centroid (Cₜ) 812b for the target breast region 808b).

At 412c, for each i-th level curve, the foregrounds from the binary masks of reference breast region 808a is flipped to overlay the foreground from the binary mask of the target breast region 808b, and to generate a composite of the two overlays 804d.

For example, as shown in image 802d of FIG. 8D, the flipping can occur with reference to the median line 702, defined with respect to the sternal notch key point 650c. In more detail, the distance 814—along an axis orthogonal to the median line 702—is initially determined, as between the median line 702 and the reference centroid (Cᵣ) 812a. Subsequently, the area within the reference bounding box 810a is flipped, across the median line 702, by an equal distance 814 such as to overlay the target bounding box 810b. As shown in image 804d of FIG. 8D, this causes an overlay between the reference bounding box 810a and the target bounding box 810b. The software may then determine a discrepancy between the reference and target breast regions 808, for that i-th level curve, based on the overlayed images.

For example, as shown in image 804d, a common overlap area 816 may be determined between the target and reference foreground breast regions 808. This common overlap area 816 indicates the area where the morphology (i.e., shape and size) of the target breast is identical to the reference breast, for that i-th level curve. Further, a non-overlap area 816 may also be determined, as between the target and reference foreground breast regions 808. The non-overlap area can indicate the area where the morphology is different between the target and reference breast regions. For example, the non-overlap area can include a dissimilarity area 818a due to false negatives and area 818b due to false positives. The negative dissimilarity area 818a is the area which exists in the reference breast region 808a but is absent in the target breast region 808b. In contrast, the positive dissimilarity area 818b is the area which exists in the target breast region 808b but is absent in the reference breast region 808a. Accordingly, the software program can not only detect the presence of non-overlap areas between the breast regions at every level, but also the shape, size, location and nature of the non-overlap (i.e., dissimilar) areas.

Act 412c is repeated for successive level-curves, and for all N-levels. For example, as shown in FIG. 8E, for each of the i-th (image 802e), i-th+1 (image 804e), i-th+2 (image 806e) and i-th+3 (image 808e) level curves, a respective flipped overlayed region 820a-820d may be generated.

While act 412c is described with respect to the right breast iso-contour being selected as the “reference” iso-contour—as stated previously, the “reference” iso-contour may also be any other extraneous surface. In these cases, act 412c may be simply performed by overlaying any other foreground reference region over the target foreground reference region, for each i-th level curve, as described above, to determine the level of dissimilarity.

Referring back to FIG. 4C, at 414c, the flipped overlay regions—for each i-th level curve—may be vertically stacked in sequential order to visualize the aggregate volumetric and structural discrepancies between the target and reference iso-contours. For example, FIG. 8F shows a stacked visualization of the overlay regions for the aggregate N-levels, i.e., curves 804a-804d.

In some cases, the visualization generated at 414c may be displayed, or output, on a display interface 202e of a user device 108 (FIG. 2A) or display 204e associated with computing terminal 112 (FIG. 2B). In some cases, a user may interact with the visualization. For example, using the user input interfaces 202f or 204f, a user may, for example, rotate the 3D visualizations to better observe various overlap and non-overlap areas, and from different viewing perspectives. The displayed visualization may also be updated in response to user inputs. For example, a user may wish to observe overlay regions for one or more specific level curves. Accordingly, the user may input a selection, via the input interfaces, indicating one or more level curves to view in more detail. For example, this can be a computer mouse or a touch display selection. In response, the system may update the visualization to only display, or otherwise emphasize, the selected level curves, i.e., either individually, stacked-up, or in any other presentation form. The user may also “flip” between level curves using the input interface. In this manner, the user may observe, in more precision, the overlap and non-overlap areas for selected level curves. This, in turn, may enable, for example, a surgeon to observe and/or adjust the morphology for a target breast, and with greater accuracy.

At 416c, in at least some embodiments, one or more dissimilarity metrics can be determined. The dissimilarity metrics can quantitate the level of dissimilarity between the target and reference breast regions. In cases where the reference breast and the target breasts is one of the subject's breasts—such that the objective is to determine the asymmetry between the subject's breasts—the dissimilarity metric can also provide a measure of the degree of asymmetry between the two breasts. In at least one embodiment, a Jaccard Index may be determined for each i-th level curve. The Jaccard Index, also known, as the Intersection-over-Union (IoU), is used to quantitatively measure the dissimilarity between each i-th level reference and target curve, and is determined in accordance with Equation (1):

$${IoU}_{i} = \frac{Area of Overlap}{Area of Union} = \frac{\vert {A}_{i} ⋂ {B}_{i} \vert}{\vert {A}_{i} ⋃ {B}_{i} \vert} = \frac{\vert {A}_{i} ⋂ {B}_{i} \vert}{\vert {A}_{i} \vert + \vert {B}_{i} \vert - \vert {A}_{i} ⋂ {B}_{i} \vert} \qquad ( 1 )$$

wherein “A” is the foreground of reference breast region 808a, and “B” is the foreground of target breast region 808b, for the i-th level-curve (i=1, 2, 3 . . . N, wherein N is the last level curve within the breast boundary).

The overall morphological dissimilarity, across all N-level curves—and as between the two breast mounds—may then be determined according to Equation (2), which averages the IOU for all i-level curves to obtain the mean IoU (mIoU):

$$mIoU = \frac{1}{N} * {\sum }_{i = 1}^{N} {IoU}_{i} \qquad ( 2 )$$

Further metrics that may be determined include the ratio of breast volumes (Vᵣ), which may be determined in accordance with Equation (3):

$${V}_{r} = \frac{min ( {V}_{T} , {V}_{R} )}{max ( {V}_{T} , {V}_{R} )} \qquad ( 3 )$$

wherein V_{T} is the volume of the target breast and V_{R} is the volume of the reference breast (i.e., as determined at act 412b of method 400b).

A novel metric has been additionally realized, which is referenced herein as the “Volumetric and Iso-contour based Morphological Asymmetry” (VIMA) score. The VIMA score may indicate the degree of asymmetry between the two breasts, and is determined in accordance with Equation (4):

$$VIMA = 1 - [ λ * mIoU + ( 1 - λ ) * {V}_{r} ] \qquad ( 4 )$$

wherein λ is empirically determined to be 0.75. In particular, the VIMA score makes use of the convex combination of: (a) the mean Jaccard Index of the level-curves from the 3D iso-contour; and (b) the ratios of pseudo-volumes, from 3D surface scans of the breast. In this sense, the VIMA score represents a weighted combination of the dissimilarity from the totality of 3D shape and the volume ratios. Accordingly, as contrasted to other metrics, the VIMA score can identify asymmetry cases, not only where the breast mounds have different volumes, but also cases where the breast mounds have identical volumes but different 3D shapes or morphologies. In general, the larger the VIMA value in Equation (4), the greater the degree of asymmetry between two breast profiles.

Referring back to act 310 of FIG. 3, as previously stated—an output may be generated based on the results of methods 400a-400c. For example, a graphic display may be generated as shown in image 800f of FIG. 8F, which is a visualization of the aggregate volumetric and structural discrepancies between the target and reference iso-contours. This can include both a 3D visualization 802f and/or a 2D projection on the XY plane 804f. In some cases, the overlap regions 816, as well as the false-negative and false-positive regions 816a, 816b can be indicated with different visual indicia (i.e., different color coding).

In this manner, the output visualization may allow a user (i.e., medical practitioner) to observe the aggregate differences between the target and reference breast iso-contours, and the precise location, size and shape of any dissimilarity. This, in turn, provides an illustrative or visualized guide for the benefit of the user before, during and after the surgical operation. The overlap regions 816 may indicate areas of the target breast which do not require modification, while the regions 818a or 818b indicate areas where tissue mass should be added or removed, respectively, to better conform the target breast morphology to the reference breast morphology, i.e., for decreasing asymmetry.

In various cases, as noted in method 300 of FIG. 3, this visualization 800f of FIG. 8F may be updated in real-time, or near real-time, as adjustments are made, i.e., during surgery. This, in turn, acts as a real-time or near real-time guide during surgery to guide modification of the target breast morphology vis-à-vis the reference breast morphology.

Graphical visualization 800f may also include other information, including: (i) the volumetric difference between the target and reference breast iso-contours 806f (i.e., as determined at 412b). This may be expressed, for example, in units of cubic centimeter (cc); (ii) the width 808f and height 810f for the regions with dissimilarity (i.e., based on the linear geometric measurements at 410b in FIG. 4B); as well as (iii) various other dissimilarity metrics, as determined at act 416c of method 400c.

Reference is now made back to FIG. 2, which shows simplified hardware/software block diagrams of example embodiments of the user device 108 and the external computing terminal 110 and/or external server 110.

As shown, the user device 108 generally includes a processor 202a coupled to one or more of a memory 202b, one or imaging sensor(s) 202c, a communication interface 202d, a display 202e and a user input interface 202f.

Processor 202a is a computer processor, such as a general-purpose microprocessor. In some other cases, processor 202a may be a field programmable gate array, application specific integrated circuit, microcontroller, or other suitable computer processor. In some cases, processor 202a may comprise multiple processors.

Processor 202a is coupled, via a computer data bus, to memory 202b. Memory 202b may include both volatile and non-volatile memory. Non-volatile memory stores computer programs consisting of computer-executable instructions, which may be loaded into volatile memory for execution by processor 202a as needed. It will be understood by those of skill in the art that references herein to user device 108 as carrying out a function or acting in a particular way imply that processor 202a is executing instructions (e.g., a software program) stored in memory 202b and possibly transmitting or receiving inputs and outputs via one or more interface. Memory 202b may also store data input to, or output from, processor 202a in the course of executing the computer-executable instructions. As noted above, memory 202b may also store the scanning software application 202f, which may be used to operate imaging sensors 202c, i.e., time-of-flight (ToF) sensors.

Communication interface 202d is one or more data network interface, such as an IEEE 802.3 or IEEE 802.11 interface, for communication over a network.

Display 202e is a suitable display for outputting information and data as needed by various computer programs.

Input interface 202f may be, for example, a keyboard, mouse, etc. In some cases, display 202e may act as an input interface 202f where the display 202e is a touch-screen display.

External computing terminal 112 and/or external server 110 (collectively 204 in FIG. 2B) may also comprise a processor 204a in communication with a memory 204b, communication interface 204c, display 204e and input interface 204f. Memory 204b may store the data processing software 204d (i.e., a breast morphology assessment software 204d).

While the above description provides examples of the embodiments, it will be appreciated that some features and/or functions of the described embodiments are susceptible to modification without departing from the spirit and principles of operation of the described embodiments. Accordingly, what has been described above has been intended to be illustrative of the invention and non-limiting and it will be understood by persons skilled in the art that other variants and modifications may be made without departing from the scope of the invention as defined in the claims appended hereto. The scope of the claims should not be limited by the preferred embodiments and examples but should be given the broadest interpretation consistent with the description as a whole.

1. A method for automatic assessment of breast morphology, comprising:
receiving three-dimensional (3D) sensor data of a breast area;
processing the 3D sensor data to determine one or more breast morphology parameters in respect of one or more breasts in the breast area, wherein the processing comprises:
generating a 2D image projection of a curvature response of the 3D sensor data;
detecting, in the 2D image, one or more key points;
estimating at least one 2D breast region boundary based on the one or more key points;
extrapolating the at least one 2D breast boundary region into 3D space to project the at least one breast boundary region onto a 3D image of the breast area;
determining the one or more breast morphology parameters based on the at least one breast region boundary defined in one or more of the 2D and 3D images of the breast area; and
generating an output comprising the one or more breast morphology parameters.

2. The method of claim 1, initially comprising:
applying simultaneous localization and mapping (SLAM) to the 3D sensor data to generate point cloud data;
processing the point cloud data to generate a 3D mesh image of the breast area, the 3D mesh image corresponding to the 3D image of the breast area; and
generating the 2D curvature tensor field image based on the 3D mesh image.

3. The method of claim 1, wherein the key points correspond to a position location of one or more of a right nipple areolar complex (NAC), a left NAC and a sternal notch.

4. The method of claim 3, wherein detecting the one or more key points in the 2D image comprises:
bisecting the 2D image to generate a right sub-image and a left sub-image;
within each sub-image, determining the point of maximum curvature as corresponding to the right NAC and left NAC key points, respectively;
cropping the 2D image to generate a cropped image, wherein the bottom left and right corners of the cropped image are aligned with the right and left NAC key points, respectively; and
within the cropped image, determining the point of maximum curvature as corresponding sternal notch (SN) key point.

5. The method of claim 4, wherein estimating at least one 2D breast boundary region based on the one or more key points comprises:
identifying a respective higher curvature region around each of the right and left NAC key points corresponding to higher curvature response; and
fitting an ellipse around the respective NAC key point and the high curvature region, wherein the ellipse fitted around the high curvature region including the right NAC key point defines the right breast region boundary, and the ellipse fitted around the high curvature region including the left NAC key point defines the left breast region boundary.

6. (canceled)

7. The method of claim 1, wherein after determining one or more breast morphology parameters, the method further comprises determining a dissimilarity between a current breast morphology and a desired breast morphology, comprising:
generating an iso-contour for at least one breast region;
generating N-level curves within the breast region boundary of the at least one breast region;
for each i-th level curve of the N-level curves:
applying a binary mask to generate a foreground region corresponding to the location of the breast region;
estimating a bounding box around the foreground region, and a box centroid, to generate a target foreground region; and
overlaying a reference foreground region over the target foreground region to generate an overlayed region comprising an overlap area and one or more non-overlap areas, wherein the reference foreground region is associated with the desired breast morphology and the target foregoing region is associated with the current breast morphology.

8. The method of claim 7, wherein the non-overlap areas comprise one or more of false negative areas and false positive areas, wherein the false negative areas are areas which exist in the reference foreground region and not in the target foreground region, and false positive areas are areas which exist in the target foreground region and not in the reference foreground region.

9. The method of claim 7, further comprising stacking the overlayed regions for each successive i-th level curve to generate a 3D visualization of the overlayed regions.

10. The method of claim 7, wherein determining the pseudo-volume of a breast region comprises: adding the areas within the iso-contours from all N-level curves.

11. (canceled)

12. The method of claim 7, further comprising determine a degree of asymmetry between the breasts, wherein for each i-th level curve, the method comprises:
selecting the foreground region for one of the breast regions as being the target foreground region and reference foreground region;
flipping the reference foreground region, across a median line defined with respect to the sternal notch key point, to overlay the target foreground region.

13. The method of claim 12, further comprising determining a “Volumetric and Iso-contour based Morphological Asymmetry” (VIMA) score according to the equation:
$$VIMA = 1 - [ λ * mIoU + ( 1 - λ ) * {V}_{r} ]$$
wherein λ is an empirically determined variable, mIoU is a mean intersection over union (IoU) determined across all N-level curves and Vᵣ is a ratio of breast mound volumes.

14. (canceled)

15. (canceled)

16. (canceled)

17. (canceled)

18. (canceled)

19. The method of claim 1, wherein the output comprises a graphical output.

20. The method of claim 19, wherein the graphical output comprises a 3D visualization of the stacking of the overlayed regions for each successive i-th level curve.

21. The method of claim 20, wherein the 3D visualization comprises, for each overlayed region in each i-th level curve, different visual indicia for the overlap area and the one or more non-overlap areas.

22. The method of claim 21, wherein the 3D visualization comprises, for each overlayed region in each i-th level curve, different visual indicia for each of the false negative areas and the false positive areas.

23. The method of claim 21, wherein the different visual indicia correspond to a different color or shading schemes.

24. The method of claim 19, wherein the output is generated on a display interface associated with a user device or a remote computer terminal.

25. The method of claim 24, further comprising:
receiving, via an input interface of the user device, a selection of one of the level curves in the 3D visualization; and
updating, on the display interface, the visualization to show visual indicia for the overlap and non-overlap areas for that level curve.

26. A system for automatic assessment of breast morphology, comprising:
at least one 3D image sensor for generating 3D sensor data; and
at least one processor configured to perform the method of claim 1.

27. A non-transitory computer readable medium storing computer executable instructions, which upon execution by at least one processor, cause the at least one processor to perform the method of claim 1.
